(19) United States
(12) Patent Application Publication
(10) Pub. No.: US 2019/0254567 A1
(43) Pub. Date: Aug. 22, 2019

(54) DEVICE FOR MEASURING RESPIRATORY PARAMETERS OF A PATIENT

(71) Applicant: CIPLA LIMITED, Maharashtra, Mumbai (IN)

(72) Inventors: Vidur MALHOTRA, Maharashtra Mumbai (IN); Srinivasan JANARDHANAN, Virugambakkam, Chennai (IN)

(21) Appl. No.: 16/328,945

(22) PCT Filed: Sep. 2, 2017
(86) PCT No.: PCT/IN2017/050378

§ 371 Date: Feb. 27, 2019

(30) Foreign Application Priority Data
Sep. 3, 2016 (IN) 201621030207

(51) Int. Cl. A61B 5/087

(52) U.S. Cl. CPC A61B 5/087; A61B 5/742; A61B 2562/0247

(57) ABSTRACT

The present invention relates to a device for measuring respiratory parameters of a patient. In one embodiment, the device 100 comprises a flow tube 101 and a body 102. The flow tube 101 is a hollow tube comprising: a first end 103, a second end 104 and at least one tube portion 105 (represented by dotted lines) wherein the tube portion is having a geometric construction capable of altering the rate/direction of air flow passing through the flow tube, thereby introducing a differential pressure between the first end 103 and the second end 104.

### TECHNICAL FIELD

The present invention relates to a device for measuring respiratory parameters of a patient.

### BACKGROUND

Prior art discloses various devices such as peak flow meters, spirometers for measuring breath parameter(s) of a patient. At one end of the spectrum is the spirometer that gives a full graph of the complete breath cycle of the user. However, a spirometer is typically very expensive and it is not possible for every patient to have a personal spirometer at home. At the opposite end of the spectrum is the mechanical peak flow meter that measures only one parameter of the breath cycle of the user i.e. the peak flow rate of exhalation. Being purely mechanical in construction, a mechanical peak flow meter is relatively inexpensive to produce and is affordable to patients who wish to keep a check on their lung function. However as the mechanical peak flow meter measure only one parameter and there is no immediate and easy method to compare results over various times, some patients may find it less attractive.

Few other options exist in the market known as electronic peak flow meters that fits in between the spectrum. However, such electronic peak flow meters employ a slew of technologies and vary widely on cost and ease of use. In addition, most of the electronic peak flow meters/spirometers available in the market use some kind of obstruction to measure the flow rate. The obstruction could be in the form of turbine blades or a venturi or a mesh to obstruct the flow of air. Over time, saliva and mucus develops on these parts and cleaning may become difficult.

Therefore, there is an unmet need for a device that is cheaper to manufacture than a professional spirometer and which provides reasonably accurate readings. Further, such a device, unlike a traditional peak flow meter, should be able to measure a couple of other important parameters in addition to the regular peak flow reading i.e. Forced Expiratory Volume in 1 second (FEV1) and Forced Vital Capacity (FVC). Furthermore, such a device should be reliable and easy to use.

### SUMMARY OF THE INVENTION

The present invention relates to a device for measuring respiratory parameters of a patient. In one embodiment, the device comprises a flow tube and a body. The flow tube is a hollow tube comprising: a first end, a second end and at least one tube portion wherein the tube portion is having a geometric construction capable of changing the rate/direction of airflow passing through the flow tube thereby creating a differential pressure between said first end and said second end. In one aspect of the invention, the ratio of area of said first end and the second end is pre-defined.

These and other aspects as well as advantages will be more clearly understood from the following detailed description taken in conjugation with the accompanying drawings.

### BRIEF DESCRIPTION OF THE ACCOMPANYING DRAWINGS

To further clarify advantages and aspects of the invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings in accordance with various embodiments of the invention, wherein:

FIG. 1 illustrates schematically an isometric view of the device 100 respectively, in accordance with an embodiment of the present invention.

FIGS. 2(a) and 2(b) illustrate schematically components in the body 102 of the device 100, in accordance with the embodiment of the present invention.

FIGS. 3(a), 3(b) and 3(c) illustrate schematically the front, side and bottom views of a flow tube 101, in accordance with an embodiment of the present invention.

FIGS. 4(a), 4(b) and 4(c) illustrate schematically the front, side and bottom views of a flow tube 101 respectively, in accordance with another embodiment of the present invention.

FIGS. 5(a), 5(b) and 5(c) illustrate schematically the front, side and bottom views of a flow tube 101 respectively, in accordance with another embodiment of the present invention.

FIGS. 6(a), 6(b) and 6(c) illustrate schematically the front, side and bottom views of a flow tube 101 respectively, in accordance with another embodiment of the present invention.

FIGS. 7(a) and 7(b) illustrate a perspective view and a front view of an exemplary device 100, in accordance with an embodiment of the present invention.

FIGS. 8(a) and 8(b) illustrates a cross- sectional view of an exemplary device 100, in accordance with an embodiment of the present invention.

It may be noted that to the extent possible, like reference numerals may have been used to represent like elements in the drawings. Further, those of ordinary skill in the art will appreciate that elements in the drawings are illustrated for simplicity and may not have been necessarily drawn to scale. For example, the dimensions of some of the elements in the drawings may be exaggerated relative to other elements to help to improve understanding of aspects of the invention. Furthermore, one or more elements may have been represented in the drawings by conventional symbols, and the drawings may show only those specific details that are pertinent to understanding the embodiments of the invention so as not to obscure the drawings with details that will be readily apparent to those of ordinary skill in the art having benefit of the description herein.

### DETAILED DESCRIPTION

It should be understood at the outset that although illustrative implementations of the embodiments of the present disclosure are illustrated below, the present invention may be implemented using any number of techniques, whether currently known or in existence. The present disclosure should in no way be limited to the illustrative implementations, drawings, and techniques illustrated below, including the exemplary design and implementation illustrated and described herein.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments, to one embodiment, to several embodiments, or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the invention.

More specifically, any terms used herein such as but not limited to “includes,” “comprises,” “has,” “consists,” and grammatical variants thereof do not specify an exact limitation or restriction and certainly do not exclude the possible addition of one or more features or elements, unless otherwise stated, and furthermore must not be taken to exclude the possible removal of one or more of the listed features and elements, unless otherwise stated with the limiting language “must comprise” or “needs to include.” Whether or not a certain feature or element was limited to being used only once, either way it may still be referred to as “one or more features” or “one or more elements” or “at least one feature” or “at least one element.” Furthermore, the use of the terms “one or more” or “at least one” feature or element do not preclude there being none of that feature or element, unless otherwise specified by limiting language such as “there needs to be one or more . . . ” or “one or more element is required.” Unless otherwise defined, all terms, and especially any technical and/or scientific terms, used herein may be taken to have the same meaning as commonly understood by one having an ordinary skill in the art. Reference is made herein to some “embodiments.” It should be understood that an embodiment is an example of a possible implementation of any features and/or elements. Some embodiments have been described for the purpose of illuminating one or more of the potential ways in which the specific features and/or elements of the invention fulfil the requirements of uniqueness, utility and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do not necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

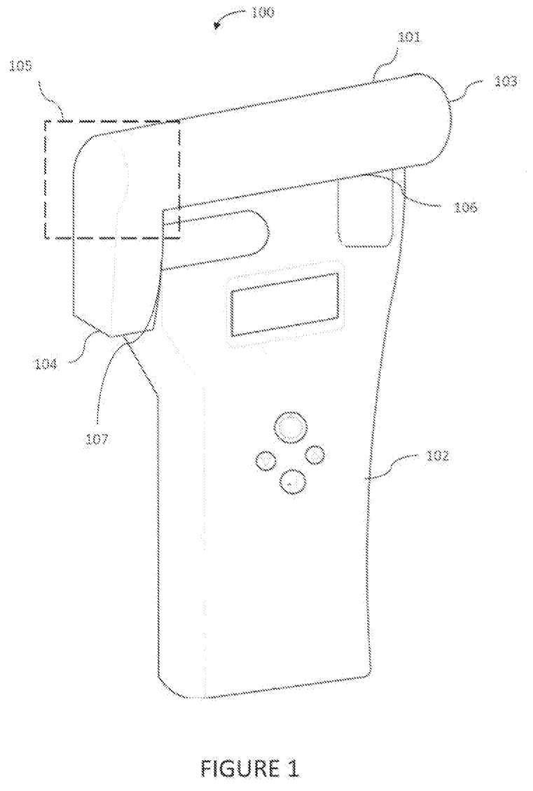

FIG. 1 illustrates an isometric view of the device 100, in accordance with an embodiment of the present invention.

As illustrated, the device 100 comprises a flow tube 101 and a body 102. The flow tube 101 is a hollow tube comprising: a first end 103, a second end 104 and at least one tube portion 105 (represented by dotted lines) wherein the tube portion 105 is having a geometric construction capable of changing the rate and/or direction of air flow passing through the flow tube thereby introducing a pressure difference between the first end 103 and the second end 104.

In one aspect of the present invention, the ratio of area (A1) of said first end and the area (A2) of the second end is pre-defined. In another aspect of the invention, the flow tube 101 comprises at least a first sensor located at a first position 106 proximate to the first end 103 and at least a second sensor located at a second position 107 proximate to the second end 104. The said first sensor and said second sensor are used to measure the air flow rates proximate to the first end 103 and the second end 104. The difference in the air flow rates is indicative of pressure difference at both the ends and is used to calculate one or more respiratory parameters of a patient exhaling into the first end of the flow tube 101. In yet another aspect of the invention, the flow tube 101 comprises a first conduit located at a first position 106 proximate to the first end 103 and a second conduit located at a second position 107 proximate to the second end 104. The said first conduit and the second conduit transfer a portion of air exhaled/inhaled by the patient to at least one pressure differential sensor, said pressure differential sensor adapted for measuring a pressure difference there between which is indicative of pressure differential between the first end 103 and the second end 104. The said pressure difference is used to calculate one or more respiratory parameters of a patient.

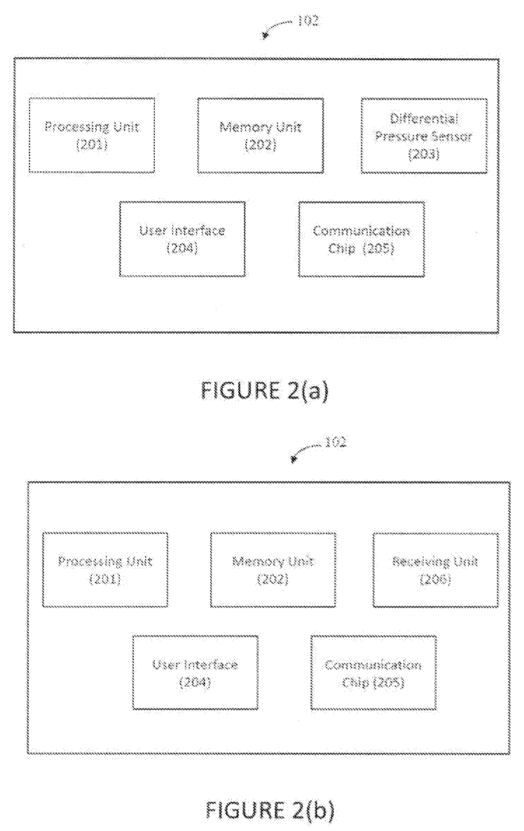

The body of the device has a shape and configuration that can be easily held by the patient while exhaling/inhaling into the first end of the flow tube 101. The components housed in body 102 are explained in detail in FIGS. 2(a) and 2(b).

It is well known to persons skilled in the art that reducing the said ratio (A1/A2) results in substantial reduction in the accuracy level of test results. Therefore, generally the said ratio (A1/A2) is set above 4.0. However, as the first end 103 of the device is to be used by the patient for exhaling and has standard dimensions that cannot be altered substantially, the second end 104 is substantially reduced to maintain the value greater than or equal to 4.0. The substantial reduction in the area of the second end generates backpressure, which causes a fundamental error in the reading of patient's breath parameters.

The present invention solves the problem by means of the tube portion, as illustrated in FIGS. 1, 3, 4, 5, and 6 that reduces the lower threshold value of ratio A1/A2 to well below 4.0, without compromising on the accuracy. More particularly, the decrease in ratio of A1/A2 to as low as 2.6 further allow substantial increase in the area of A2 due to which the problem of backpressure is substantially reduced.

According to one implementation of the present invention, the ratio of area A1 of the first end 103 and area A2 of the second end 104 is in a range of 2.0-4.2. According to another implementation of the present invention, the ratio of area A1 of the first end 103 and area A2 of the second end 104 is in a range of 2.2-3.0. According to yet another implementation of the present invention, the ratio of area A1 of the first end 103 and area A2 of the second end 104 includes a minimum value 2.6. The ranges and/or values of the ratio of (A1/A2) indicated in the present disclosure allow overcoming the problem of backpressure in the flow tube 101, as described above.

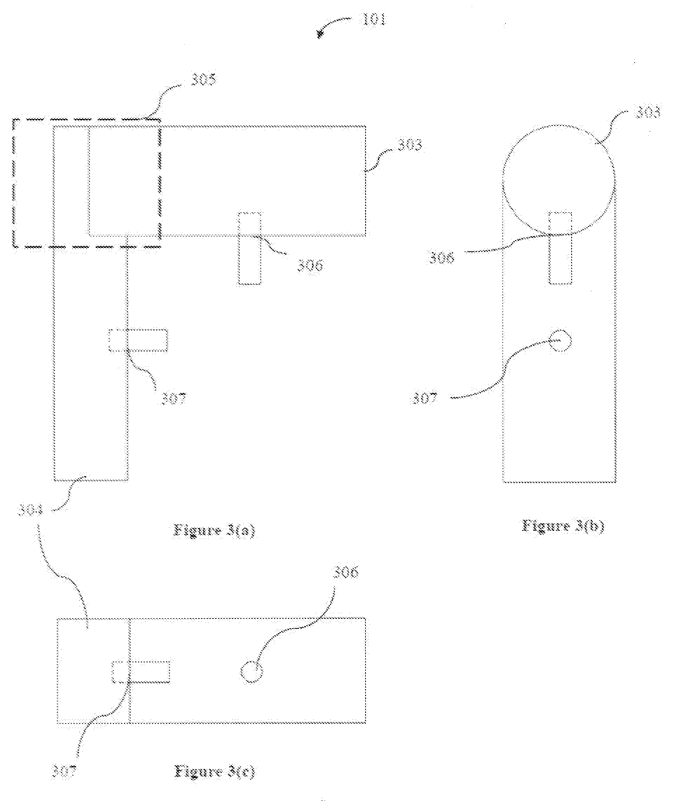

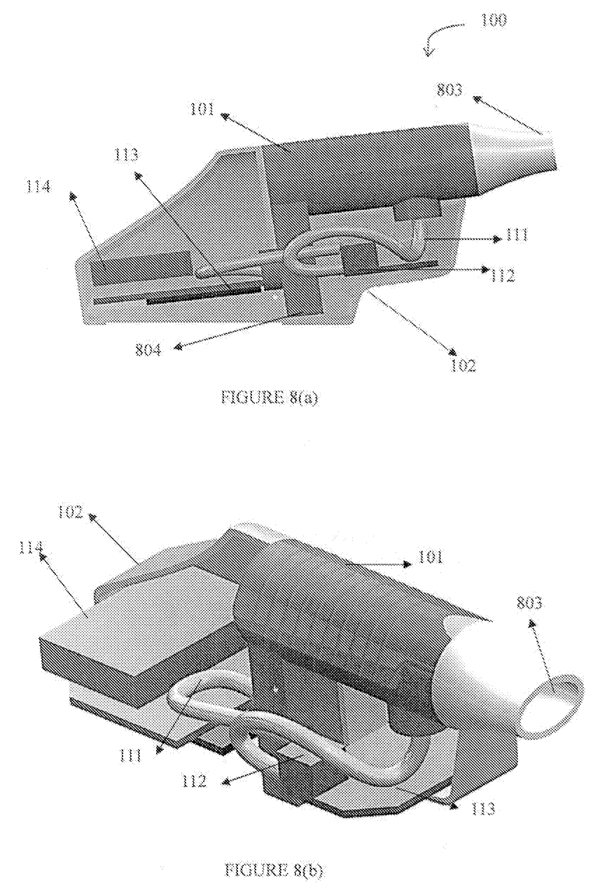

In another embodiment, the first end 103 and second end 104 may be of same shape or different shapes. The shape of the first end 103 and the second end 104 may be from a group comprising: elliptical, circular, rectangular and the likes. In one example, the shape of the first end 103 and the second end 104 include a circular shape, for example end 303 as seen in FIG. 3. In another example, as shown in FIGS. 8(a) and 8(b), the shape of a first end 803 of the flow tube 101 is that of an elliptical frustum. In another example as seen in FIG. 1, the shape of the end 104 of the flow tube 101 include a rectangular shape. Also, as seen in FIG. 8(a), a second end 804 of the flow tube 101 includes a rectangular shape.

In one embodiment of the present invention, the first end 103 and the second end 104 may be in same plane or different planes. According to some embodiments as detailed in the foregoing description, the first end and the second end of the flow tube 101 are located in planes substantially parallel to each other. According to some other embodiments as detailed in the foregoing description, the first end and the second end of the flow tube 101 are located in planes perpendicular to each other. According to yet some other embodiments as detailed in the foregoing description, the first end and the second end of the flow tube 101 are located in planes at an angle other than 90 degrees, with respect to each other.

In one embodiment of the present invention, the tube portion 105 may be continuous or abrupt. In another embodiment of the present invention, the tube portion 105 may be of varying shapes as discussed in FIGS. 3, 4, 5 and 6. In one example, the tube portion 105 is an L-shaped portion as illustrated in FIG. 3.

In yet another embodiment, the flow tube 101 is detachable from the body 102 of the device 100.

FIG. 2 schematically illustrates components in the body 102 of the device 100, in accordance with the embodiment of the present invention.

In one embodiment, as illustrated in FIG. 2(a), the body 102 of the device 100 may include one or more: a processing unit 201, a memory unit 202, a differential pressure sensor 203, a user interface 204 and a communication chip 205. The differential pressure sensor 203 receives a portion of air exhaled/inhaled by the patient via the first conduit and the second conduit (present in the flow tube) and determines the differential pressure. The differential pressure, thus determined, is provided to the processing unit 201 for further processing. The differential pressure, thus determined, may be stored in the memory unit for later processing or may be sent to an external computing device via the communication chip 205.

In another embodiment, as illustrated in FIG. 2(b), the body 102 of the device 100 may include one or more: a processing unit 201, a memory unit 202, a user interface 204, a communication chip 205 and a receiving unit 206. The receiving unit 206 is configured to receive inputs from the sensors present on the flow tube and transfer it to the processing unit 201 for further processing. In addition, the receiving unit 206 may transfer the readings to the memory unit 202 wherein it can be stored for further processing by the processing unit 201 or be transmitted to an external computing device via communication chip 205.

In both the above-discussed embodiment, the processing unit 201 may include one or more processors, microprocessors, application specific integrated circuits (ASICs), field programmable gate arrays (FPGAs), or the likes. The processing unit 201 may control the operation of the said device 100 and its components. The memory unit 202 may include a random access memory (RAM), a read only memory (ROM), and/or other type of memory to store data and instructions that may be used by the processing unit 201. The User Interface 204 may include mechanisms for inputting information to the device 100 and/or for outputting information from the device 100. Examples of input and output mechanisms might include: a speaker to receive electrical signals and output audio signals; a microphone to receive audio signals and output electrical signals; buttons (e.g., control buttons and/or keys of a keypad) to permit data and control commands to be input into the device 100; a display to output visual information such as respiratory data of the patient; a light emitting diode; a vibrator to cause the device 100 to vibrate. In one embodiment, the processor may be coupled to one or more warning indicators that may alert the user of a potential problem with the recorded respiratory function, such as the measured respiratory reading being outside a preprogrammed reference range. The communication chip 205 may include any transceiver-like mechanism that enables the device 100 to communicate with other devices and/or systems. In one example, blue-tooth functionality is added so that the data captured by the device 100 can be transferred to an external computing device to read and collate the respiratory data of the user/patient. An application corresponding to the device 100 of the present invention can also be made available in the external computing device. Further, smart medication devices may be coupled to the device 100 and/or the application corresponding to device 100 available in the computing device such that date, time and medication dispensed by said smart medication device can be collated with the respiratory data information.

Although FIGS. 2(a) and 2(b) shows a number of components of the body 102, in other implementations, the body 102 may include fewer components, different components, differently arranged components, or additional components than depicted in said FIGS. 2(a) and 2(b). Additionally or alternatively, one or more components of the body 102 may perform the tasks described as being performed by one or more other components of the device 100.

FIGS. 3(a), 3(b) and 3(c) schematically illustrate the front, side and bottom views of a flow tube 101, in accordance with an embodiment of the present invention.

As illustrated, the flow tube 101 is a hollow tube comprising: a first end 303, a second end 304 and at least one tube portion 305 (represented by dotted lines). The tube portion 305 is an L shaped portion and capable of changing the rate and/or direction of airflow passing through the flow tube 101. The first end 303 and second end 304 are located in planes perpendicular to each other. The shape of the first end is circular whereas the shape of the second end is rectangular. The first sensor/conduit is located at a first position 306 proximate to the first end 303 and the second sensor/conduit is located at a second position 307 located proximate to the second end 304 of the device 100. In addition, the ratio of area of the first end and the second end is greater than or equal to 2.6.

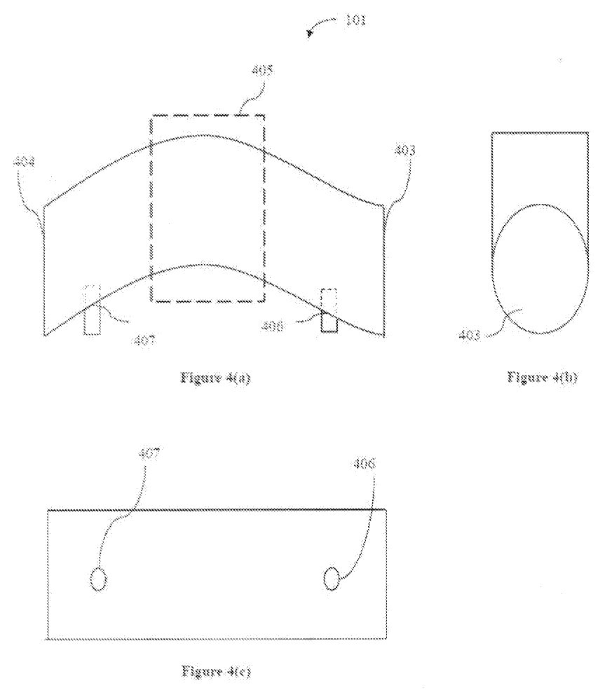

FIGS. 4(a), 4(b) and 4(c) schematically illustrate the front, side and bottom views of a flow tube 101 respectively, in accordance with an embodiment of the present invention.

As illustrated, the flow tube 101 is a hollow tube comprising: a first end 403, a second end 404 and at least one tube portion 405 (represented by dotted lines). The tube portion 405 is capable of changing the rate and/or direction of airflow passing through the flow tube. The first end 403 and second end 404 are located in planes substantially parallel to each other. The shape of the first end and the second end is circular. The first sensor/conduit is located at a first position 406 proximate to the first end 403 and the second sensor/conduit is located at a second position 407 proximate to the second end 404 of the device 100. In addition, the ratio of area of the first end and the second end is greater than or equal to 2.6.

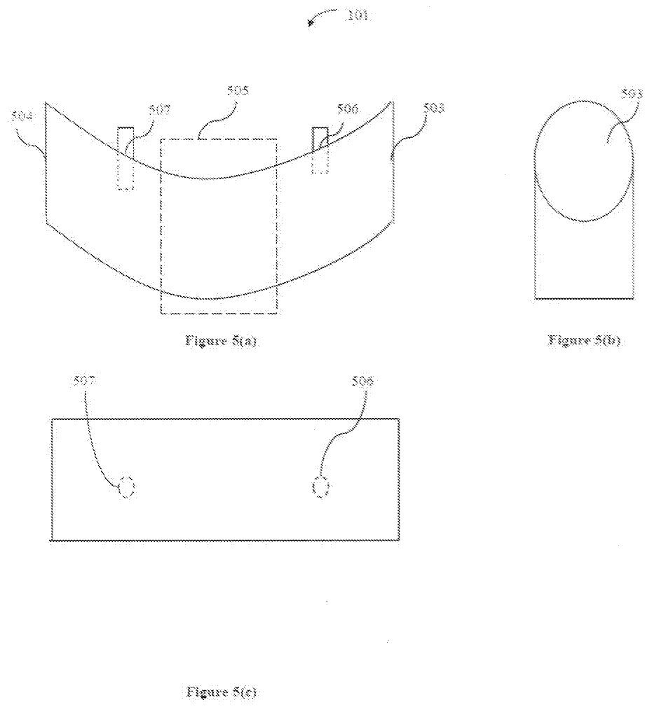

FIGS. 5(a), 5(b) and 5(c) schematically illustrate the front, side and bottom views of a flow tube 101 respectively, in accordance with an embodiment of the present invention.

As illustrated, the flow tube 101 is a hollow tube comprising: a first end 503, a second end 504 and at least one tube portion 505 (represented by dotted lines). The tube portion 505 is capable of changing the rate and/or direction of airflow passing through the flow tube. The first end 503 and second end 504 are located in planes substantially parallel to each other. The shape of the first end and the second end is circular. The first sensor/conduit is located at a first position 506 proximate to the first end 503 and the second sensor/conduit is located at a second position 507 proximate to the second end 504 of the device 100. In addition, the ratio of area of the first end and the second end is greater than or equal to 2.6.

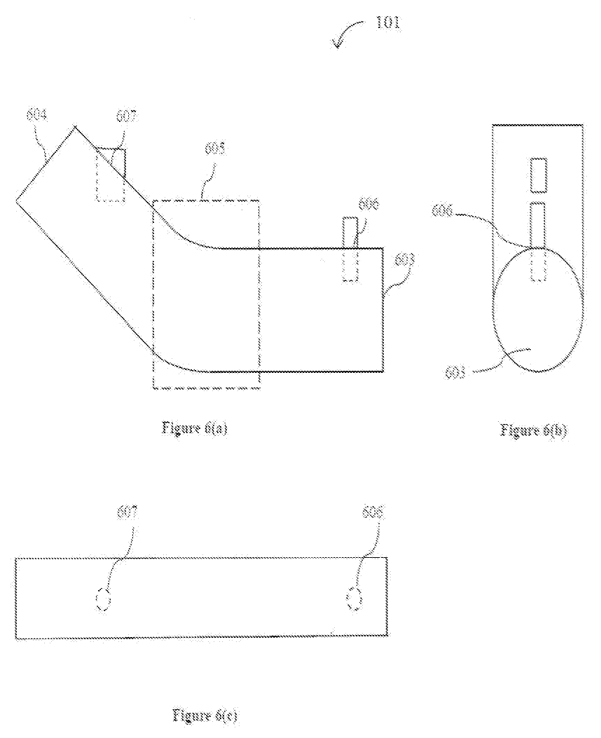

FIGS. 6(a), 6(b) and 6(c) schematically illustrate the front, side and bottom views of a flow tube 101 respectively, in accordance with an embodiment of the present invention.

As illustrated, the flow tube 101 is a hollow tube comprising: a first end 603, a second end 604 and at least one tube portion 605 (represented by dotted lines). The tube portion 605 is capable of changing the rate and/or direction of airflow passing through the flow tube. The first end 603 and second end 604 are located in different planes. The shape of the first end and the second end is circular. The first sensor/conduit is located at a first position 606 proximate to the first end 603 and the second sensor/conduit is located at a second position 607 proximate to the second end 604 of the device 100. In addition, the ratio of area of the first end and the second end is greater than or equal to 2.6.

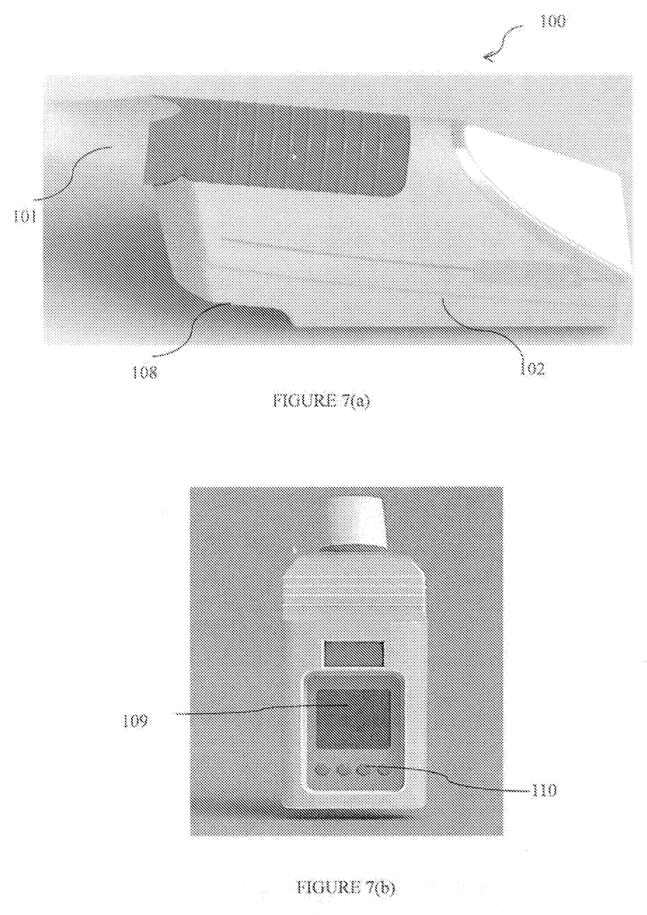

FIGS. 7(a) and 7(b) illustrate a perspective view and a front view of an exemplary device 100, in accordance with an embodiment of the present invention. The exemplary device 100 embodies the structure of the flow tube 101 as schematically illustrated in FIG. 1. For the sake of brevity, some parts of the structure of the flow tube 101, for example the tube portion 105, are not illustrated in these FIGS. 7(a) and 7(b). Further, for the sake of clarity, the reference numeral for the flow tube 101 is consistent with FIGS. 1, 3, 4, 5 and 6, and the reference numeral for the body 102 is consistent with FIG. 1.

As illustrated in FIG. 7(a), the flow tube 101 is shown connected to the body 102 of the device 100. Further, the body 102 includes a gripping potion 108 enabling easy gripping of the device 100 by the patient using his thumb.

Further, as illustrated in FIG. 7(b), the body 102 includes a display 109 to enable viewing output visual information such as respiratory data of the patient, and buttons 110 or control buttons 110 to permit user for input data and control commands into the device 100.

FIGS. 8(a) and 8(b) illustrate a cross-sectional view of an exemplary device 100, in accordance with an embodiment of the present invention. The exemplary device 100 embodies the structure of the flow tube 101 as schematically illustrated in FIG. 1. For the sake of brevity, some parts of the structure of the flow tube 101, for example the tube portion 105, are not illustrated in these FIGS. 8(a) and 8(b). Further, for the sake of clarity, the reference numeral for the flow tube 101 is consistent with FIGS. 1, 3, 4, 5 and 6, and the reference numeral for the body 102 is consistent with FIG. 1.

As illustrated in FIGS. 8(a) and 8(b), the flow tube 101 having a first end 803 and a second end 804, is shown connected to the body 102. The cross-section view further indicates exemplary embodiment of the internal components of the body 102. The internal components include conduits 111 to transfer a portion of air exhaled/inhaled by the patient to a pressure sensor 112. Further, the internal components include a printed circuit board (PCB) 113 to embed one or more electrical components disclosed above. Further, the internal components include a battery 114 for providing power supply to the device 100 for the functioning of the electrical components as described above.

Further, according to one of the implementation of the device 100 as shown in FIGS. 8(a) and 8(b), the ratio of area A1 of the first end 803 and area A2 of a second end 804 is in a range of 2.0-4.2. According to another implementation, the ratio of area A1 of the first end 803 and area A2 of the second end 804 is in a range of 2.2-3.0. According to yet another implementation, the ratio of area A1 of the first end 803 and area A2 of the second end 804 includes a minimum value 2.6.

While certain present preferred embodiments of the invention have been illustrated and described herein, it is to be understood that the invention is not limited thereto. Clearly, the invention may be otherwise variously embodied and practiced within the scope of the claims and complete specification to follow.

1. A device (100) for measuring respiratory parameters, the device (100) comprising:
a flow tube (101); and
a body (102) to be hand-held;
wherein the flow tube (101) is a hollow tube comprising:
a first end (103, 303, 403, 503, 603, 803);
a second end (104, 304, 404, 504, 604, 804);and
at least one tube portion (105, 305, 405, 505, 605) wherein the tube portion (105, 305, 405, 505, 605) is having a geometric construction capable of changing the rate and/or direction of airflow passing through the flow tube (101) thereby creating a differential pressure between the first end (103, 303, 403, 503, 603, 803) and the second end (104, 304, 404, 504, 604, 804);
wherein the device (100) further comprises at least one sensor to detect the differential pressure thus created.

2. The device (100) as claimed in claim 1 wherein a ratio of an area (A1) of the first end (103, 303, 403, 503, 603, 803) to an area (A2) of the second end (104, 304, 404, 504, 604, 804) in a range of 2.2-4.2.

3. The device (100) as claimed in claim 1 wherein a ratio of an area (A1) of the first end (103, 303, 403, 503, 603) to an area (A2) of the second end (104, 304, 404, 504, 604) is in a range of 2.2-3.0.

4. The device (100) as claimed in claim 2 wherein a ratio of an area (A1) of the first end (103, 303, 403, 503, 603, 803) to an area (A2) of the second end (104, 304, 404, 504, 604, 804) includes a minimum value of 2.6.

5. The device (100) as claimed in claim 1 wherein the first end (403, 503) and the second end (404, 504) are in planes substantially parallel to each other.

6. The device (100) as claimed in claim 1 wherein the first end (603) and the second end (604) are in planes at an angle other than 90 degrees, with respect to each other.

7. The device (100) as claimed in claim 1 wherein the first end (103, 303, 803) and the second end (104, 304, 804) are in planes perpendicular to each other.

8. The device (100) as claimed in claim 7 wherein the tube portion (105, 305) is an L-shaped portion.

9. The device (100) as claimed in claim 1, wherein the first end (103, 303, 403, 503, and 603, 803) and the second end (104, 304, 404, 504, and 604, 804) have a similar shape or different shapes.

10. The device (100) as claimed in claim 9, wherein the shape of the first end (103, 303, 403, 503, 603, 803) and the second end (104, 304, 404, 504, 604, 804) include one of:
circular shape;
rectangular shape;
elliptical shape.

11. The device (100) as claimed in claim 1 wherein the tube portion (105, 305, 405, 505, 605) is continuous or abrupt.

12. The device (100) as claimed in claim 1 wherein the at least one sensor is located in the flow tube (101).

13. The device (100) as claimed in claim 12 wherein the flow tube (101) comprises a first sensor located at a first position (106) proximate to the first end (103, 303, 403, 503, 603) and at least a second sensor located at a second position (107) proximate to the second end (104, 304, 404, 504, 604).

14. The device (100) as claimed in claim 13, wherein the body (102) includes a receiving unit (206) to receive inputs from the sensor(s) located in the flow tube (101).

15. The device (100) as claimed in claim 1 wherein the at least one sensor is located in the body (102).

16. The device (100) as claimed in claim 15 wherein the flow tube (101) comprises a first conduit located at a first position (106, 306, 406, 506, 606) proximate to the first end (103, 303, 403, 503, 603, 803) and a second conduit located at a second position (107, 307, 407, 507, 607) proximate to the second end (104, 304, 404, 504, 604, 804), wherein the first conduit and the second conduit transfer a portion of air exhaled/inhaled by the patient to the at least one sensor located in the body (102).

17. The device (100) as claimed in claim 1 wherein the flow tube (101) is detachably connected to the body (102).

18. The device (100) as claimed in claim 1, wherein the body (102) comprising:
a processing unit (201) to process the differential pressure thus detected;
a memory unit (202) to store one or more values of the differential pressure being detected on the device (100); and
a user interface (204) configured to communicate with a user of the device (100).

19. The device (100) as claimed in claim 17, further comprising a communication chip (205) to communicate with an external device.
